(19) United States
(12) Patent Application Publication
(10) Pub. No.: US 2020/0048189 A1
(43) Pub. Date: Feb. 13, 2020

(54) GUANIDINE CATALYST FOR CURABLE COMPOSITIONS

(71) Applicant: SIKA TECHNOLOGY AG, Baar (CH)

(72) Inventors: Rita CANNAS, Dübendorf (CH); Urs BURCKHARDT, Zürich (CH)

(73) Assignee: SIKA TECHNOLOGY AG, Baar (CH)

(21) Appl. No.: 16/492,976

(22) PCT Filed: Mar. 14, 2018
(86) PCT No.: PCT/EP2018/056457

§ 371 Date: Sep. 11, 2019

(30) Foreign Application Priority Data
Mar. 24, 2017 (EP) 17162783.9

(51) Int. Cl. C07C 279/16; C08G 18/10; C08G 18/18; C08G 18/48; C08G 18/72; C08G 18/73; C08G 18/75; C09D 175/08; C08G 18/50

(52) U.S. Cl. CPC C07C 279/16; C08G 18/10; C08G 18/1858; C08G 18/4866; C08G 18/722; C07C 277/08; C08G 18/755; C08G 18/758; C09D 175/08; C08G 18/5096; C08G 18/73

(57) ABSTRACT

A guanidine of the formula (I) and to the use thereof as catalyst for the crosslinking of a functional compound, especially a polymer having silane groups. The guanidine of the formula (I) described is preparable in a simple method via a polyfunctional carbodiimide adduct from the readily available raw materials, and is largely odorless at room temperature and of low toxicity. In spite of its comparatively high molecular weight, it accelerates the crosslinking of functional compounds surprisingly well, and such compositions do not have a tendency to migration-related defects such as separation, exudation or substrate soiling.

### TECHNICAL FIELD

The invention relates to guanidines and to the use thereof as catalyst for curable compositions, especially based on polymers having silane groups.

### PRIOR ART

Curable compositions play a significant role in many industrial applications, for example as adhesives, sealants or coatings. The curing thereof is brought about by crosslinking reactions which proceed via reactive groups, for example silane groups, isocyanate groups or epoxy groups, wherein these react with themselves or a coreactant following a mixing operation or through heating or through contact with moisture, and hence form a polymeric network in the composition. Acceleration of such crosslinking reactions is frequently accomplished using catalysts. These are very often substances of toxicological concern which constitute a potential hazard to users and the environment, especially after the curing of the composition, if the catalyst or degradation products thereof are released by outgassing, migration or washing-out. Compositions curable at room temperature that are based on polymers having silane groups are confronted with this problem to a significant degree.

Polymers having silane groups here are especially polyorganosiloxanes, which are commonly referred to as “silicones” or “silicone rubbers”, and organic polymers having silane groups, which are also referred to as “silane-functional polymers”, “silane-modified polymers” (SMP) or “silane-terminated polymers” (STP). The crosslinking thereof proceeds via the condensation of silanol groups to form siloxane bonds and is conventionally catalyzed by means of organotin compounds such as dialkyltin(IV) carboxylates in particular. These are notable for very high activity in relation to the silanol condensation and are very hydrolysis-resistant, but they are harmful to health and a severe water pollution hazard. They are often combined with further catalysts, mainly basic compounds, such as amines in particular, which particularly accelerate the preceding hydrolysis of the silane groups.

Because greater weight is being given to EHS aspects by professional organizations and users and because of stricter government regulation, there have been increased efforts for some time to replace organotin compounds with other catalysts of lower toxicity. For instance, organotitanates, -zirconates and -aluminates have been described as alternative metal catalysts. However, these usually have lower catalytic activity in relation to the silanol condensation and bring about much slower crosslinking. Because of their lack of hydrolysis stability, they can lose a large part of their activity in the course of storage of the composition as a result of residual moisture in the ingredients, which causes the curing to slow significantly or stop entirely.

A further known alternative to organotin compounds is highly basic nitrogen compounds from the class of the amidines and guanidines, which can be used in combination with the metal catalysts mentioned or else alone. However, many of the commonly used amidine and guanidine catalysts, such as, in particular, 1,8-diazabicyclo[5.4.0]undec-7-ene (DBU) or 1,1,3,3-tetramethylguanidine (TMG), are volatile and odorous substances that are likewise harmful to health and hazardous to the environment. Moreover, they have a tendency to migrate because of low compatibility with the composition and hence to cause separation, exudation or substrate soiling.

Further guanidine catalysts are known from WO 2015/158859, WO 2015/158860, WO 2015/158863 and WO 2016/180840. However, these catalysts are still capable of improvement with regard to their catalytic activity and/or compatibility with different polymer systems.

U.S. Pat. No. 4,192,925 describes guanidine-modified polyols and the use thereof in polyurethane foams. However, the structures disclosed are of very high molecular weight and have few active guanidine groups, which means that they are barely suitable as catalysts for curable compositions, especially those based on polymers having silane groups.

### SUMMARY OF THE INVENTION

It is therefore an object of the present invention to provide a catalyst which overcomes the disadvantages of the prior art and especially has low odor and low toxicity, has high catalytic activity for the crosslinking reaction of functional compounds and has good compatibility with curable compositions thereof. This object is achieved by a guanidine of the formula (I) as described in claim 1. The guanidine of the invention is very substantially odorless and of low toxicity. In spite of comparatively high molecular weight, it has surprisingly high catalytic activity for the crosslinking reaction of various functional compounds, especially compounds having silane groups and/or isocyanate groups, and brings about rapid curing of curable compositions thereof. The guanidine of the formula (I) is preparable in a simple process from readily obtainable raw materials via a multifunctional carbodiimide adduct. It has good miscibility with different functional compounds, especially with polymers having silane groups, and shows barely any separation or migration either before or after curing. Further aspects of the invention form the subject matter of further independent claims. Particularly preferred embodiments of the invention form the subject matter of the dependent claims.

### Ways of Executing the Invention

The invention provides a guanidine of the formula (I)

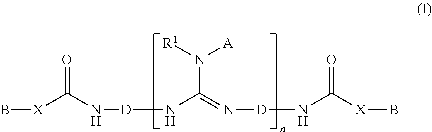

where

n is an integer from 1 to 20,

A is an alkyl, cycloalkyl or aralkyl radical which has 1 to 100 carbon atoms and optionally has one or more heteroatoms, especially oxygen or nitrogen in the form of hydroxyl, ether, amino, amidine or guanidine groups, or together with R¹ is an alkylene radical which has 2 to 6 carbon atoms and optionally contains ether oxygen or amine nitrogen and is optionally substituted,

R¹ is a hydrogen radical or is an alkyl radical which has 1 to 8 carbon atoms and optionally contains ether oxygen, or together with A is an alkylene radical which has 2 to 6 carbon atoms and optionally contains ether oxygen or amine nitrogen and is optionally substituted,

D is a divalent aliphatic or cycloaliphatic or arylaliphatic radical having 6 to 15 carbon atoms,

B is a monovalent organic radical which has an average molecular weight in the range from 150 to 5′000 g/mol and is free of hydroxyl groups, primary and secondary amino groups and mercapto groups, and

X is O or S or NR² where R² is a hydrogen radical or is an alkyl radical which has 1 to 8 carbon atoms and optionally contains ether oxygen or tertiary amine nitrogen.

A dotted line in the formulae in this document in each case represents the bond between a substituent and the corresponding molecular radical.

“Primary amino group” and “primary amine nitrogen” refer respectively to an amino group and the nitrogen atom thereof that is bonded to a single organic radical and bears two hydrogen atoms; “secondary amino group” and “secondary amine nitrogen” refer respectively to an amino group and the nitrogen atom thereof that is bonded to two organic radicals which may also together be part of a ring and bears one hydrogen atom; and “tertiary amino group” and “tertiary amine nitrogen” refer respectively to an amino group and the nitrogen atom thereof that is bonded to three organic radicals, two or three of which together may also be part of one or more rings, and does not bear any hydrogen atom.

“Functional compound” refers to a compound which cures with a suitable coreactant to give a polymeric structure and bears at least one reactive group, especially at least two reactive groups. Their reactive groups are typically electrophilic, whereas the coreactants are nucleophilic or bear nucleophilic groups.

A “curable” composition refers to one that can cure through crosslinking reactions of reactive groups present therein or attain a state of elevated mechanical strength.

The term “silane group” refers to a silyl group which is bonded to an organic radical or to a polysiloxane radical and has one to three, especially two or three, hydrolyzable substituents on the silicon atom. Particularly commonly used hydrolyzable substituents are alkoxy radicals. These silane groups are also referred to as “alkoxysilane groups”. Silane groups may also be in partly or fully hydrolyzed form.

“Silane” refers both to organoalkoxysilanes bearing one to three organic substituents on each alkoxysilane group and tetraalkoxysilanes. Silanes that bear one or more hydroxyl, isocyanato, amino or mercapto groups in addition to the silane group on an organic radical are referred to as “hydroxysilane”, “isocyanatosilane”, “aminosilane” and “mercaptosilane” respectively.

The term “organic polymer” encompasses a collective of macromolecules that are chemically homogeneous but differ in relation to degree of polymerization, molar mass and chain length, which has been prepared by a poly reaction (polymerization, polyaddition, polycondensation) and has a majority of carbon atoms in the polymer backbone, and reaction products of such a collective of macromolecules. Polymers having a polyorganosiloxane backbone (commonly referred to as “silicones”) are not organic polymers in the context of the present document.

The term “polyether having silane groups” refers to organic polymers which have silane groups and, have mainly polyether units in the polymer chain. In addition to the polyether units, urethane groups, urea groups or thiourethane groups may especially also be present. Such polyethers having silane groups may also be referred to as “polyurethanes having silane groups”.

“Molecular weight” refers to the molar mass (in g/mol) of a molecule. “Average molecular weight” is the number average Mₙ of a polydisperse mixture of oligomeric or polymeric molecules, which is typically determined by means of gel permeation chromatography (GPC) against polystyrene as standard.

“Storage-stable” or “storable” refers to a substance or composition if it can be stored at room temperature in a suitable container over a prolonged period, typically from at least 3 months up to 6 months or more, without any change in its application or use properties, especially in the viscosity and crosslinking rate, to an extent relevant for the use thereof, as a result of the storage.

“Room temperature” refers to a temperature of about 23° C.

The guanidine of the formula (I) may also be in tautomeric form in relation to the guanidine groups present. All possible tautomer forms are considered to be equivalent. In addition, it may also be in protonated form. It may likewise be in complexed form, especially with cations of zinc, iron or molybdenum.

The guanidine of the formula (I) is preferably free of hydroxyl groups. Such a guanidine is particularly storage-stable in a composition containing alkoxysilane groups.

n is preferably an integer from 1 to 10, especially from 2 to 8.

Preferably, A is an alkyl, cycloalkyl or aralkyl radical which has 1 to 50 carbon atoms and optionally has one or more hydroxyl groups, ether groups and/or amino groups, or together with R¹ is an alkylene radical which has 2 to 6 carbon atoms and optionally contains ether oxygen or tertiary amine nitrogen and is optionally substituted.

Preferably, A is free of hydroxyl groups.

More preferably, A is selected from the group consisting of methyl, ethyl, propyl, isopropyl, n-butyl, isobutyl, sec-butyl, tert-butyl, n-pentyl, isopentyl, n-hexyl, isohexyl, n-octyl, 2-ethylhexyl, n-decyl, lauryl, cocoalkyl, oleyl, cyclohexyl, benzyl, 2-methoxyethyl, 3-methoxypropyl, 3-(2-ethylhexyloxy)propyl, 3-(N,N-dimethylamino)-propyl and (ω-(alkyloxy)poly(oxyalkylene)alkyl having 1,2-oxyethylene and 1,2-oxypropylene units and a molecular weight in the range from 180 to 600 g/mol.

Among these, preference is given to A radicals having 1 to 12 carbon atoms, i.e. methyl, ethyl, propyl, isopropyl, n-butyl, isobutyl, sec-butyl, tert-butyl, n-pentyl, isopentyl, n-hexyl, isohexyl, n-octyl, 2-ethylhexyl, n-decyl, lauryl, cyclohexyl, benzyl, 2-methoxyethyl, 3-methoxypropyl, 3-(2-ethylhexyloxy)propyl or 3-(N,N-dimethyl-amino)propyl. These guanidines of the formula (I) are particularly active.

Preferably, R¹ is a hydrogen radical or is methyl, ethyl, n-propyl or n-butyl, especially a hydrogen radical or methyl.

Further preferably, A and R¹ together are 1,4-butylene, 1,5-pentylene, 3-oxa-1,5-pentylene or N-methyl-3-aza-1,5-pentylene.

Preferably, D is selected from the group consisting of 2-methyl-1,5-pentylene, 1,6-hexylene, 2,2(4),4-trimethyl-1,6-hexamethylene, (1,5,5-tri-methylcyclohexan-1-yl)methane-1,3, 1,3-cyclohexylenebis(methylene), 1,4-cyclohexylenebis(methylene), 1,3-phenylenebis(methylene) and (methylenedi-cyclohexyl)-4,4′. Such a guanidine of the formula (I) is derived from commercially available aliphatic diisocyanates and is thus particularly readily available.

More particularly, D is 1,6-hexylene, (1,5,5-trimethylcyclohexan-1-yl)methane-1,3 or (methylenedicyclohexyl)-4,4′.

More preferably, D is (methylenedicyclohexyl)-4,4′.

Preferably, B is a monovalent organic radical which has an average molecular weight in the range from 150 to 2′000 g/mol and especially has ether groups.

More preferably, B is lauryl, cocoalkyl, oleyl, 2-(ω-(alkyloxy)poly-(oxyethylene))ethyl, 2-((ω-(alkyloxy)poly(oxyethyleneoxypropylene))ethyl, 2-(ω-(alkyloxy)poly(oxyethylene-oxypropylene))propyl or 2-(ω-(alkyloxy)poly(oxypropylene))propyl, where alkyl represents methyl, ethyl, butyl, 2-ethylhexyl, lauryl, cocoalkyl or oleyl and the polymeric radicals have an average molecular weight in the range from 150 to 2′000 g/mol, especially 200 to 1′000 g/mol.

Among these, preference is given to the polyether radicals. Such a guanidine of the formula (I) has particularly good compatibility with polyether polymers and hence is of particularly good suitability as catalyst for polyether-based functional compounds or curable compositions.

Preferably, X is O or NR², especially O.

Preferably, R² is a hydrogen radical or is methyl, especially a hydrogen radical. More preferably, X is O and B is 2-(ω-(alkyloxy)poly(oxyethylene))ethyl, 2-(ω-(alkyloxy)poly(oxyethyleneoxypropylene))ethyl, 2-(ω-(alkyloxy)-poly(oxyethyleneoxypropylene))propyl or 2-(ω-(alkyloxy)poly(oxypropylene))-propyl, where alkyl represents methyl, ethyl, butyl, 2-ethylhexyl, lauryl, cocoalkyl or oleyl and these radicals have an average molecular weight in the range from 150 to 2′000 g/mol, especially 200 to 1′000 g/mol. Such a guanidine of the formula (I) is usually liquid at room temperature and of comparatively low viscosity, has particularly good compatibility with polyether polymers and hence is particularly suitable as catalyst for polyether-based functional compounds or curable compositions.

Preferably, the guanidine of the formula (I) has an average value of n in the range from 1.1 to 6, preferably 1.5 to 4, especially 1.8 to 3. Such a guanidine of the formula (I) has comparatively low viscosity and is very catalytically active.

The invention further provides a process for preparing the guanidine of the formula (I) by reacting a carbodiimide adduct of the formula (II) with at least one amine of the formula (III)

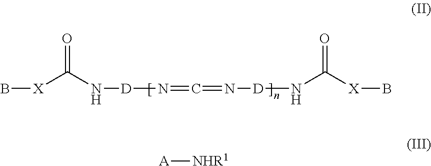

where n, A, R¹, D, B and X have the definitions already given.

This reaction is preferably conducted at a temperature in the range from 20 to 160° C., especially 40 to 140° C., preferably 60 to 120° C., optionally in the presence of at least one catalyst.

Preference is given to conducting the reaction in the absence of solvents. Preference is given to choosing a roughly stoichiometric ratio between the amine of the formula (III) and the carbodiimide groups of the carbodiimide adduct of the formula (II), such that most of the carbodiimide groups are converted.

Preference is given to using the resultant guanidine of the formula (I) without further processing or purification.

Suitable amines of the formula (III) are especially methylamine, dimethylamine, ethylamine, diethylamine, propylamine, isopropylamine, butylamine, dibutylamine, isobutylamine, sec-butylamine, tert-butylamine, n-pentylamine, isopentylamine, n-hexylamine, isohexylamine, n-octylamine, 2-ethylhexylamine, n-decylamine, laurylamine, cocoalkylamine, oleylamine, cyclohexylamine, N-methylcyclohexylamine, benzylamine, N-methylbenzylamine, 2-methoxyethylamine, 3-methoxypropylamine, 3-(2-ethylhexyloxy)propylamine, 3-(N,N-dimethylamino)propylamine, pyrrolidine, piperidine, morpholine, N-methylpiperazine or ω-(alkyloxy)poly-(oxyalkylene)alkylamine having 1,2-oxyethylene and 1,2-oxypropylene units and a molecular weight in the range from 180 to 600 g/mol, especially Jeffamine® M-600 (from Huntsman).

A suitable carbodiimide adduct of the formula (II) is especially obtained from the reaction of at least one carbodiimide of the formula (IV) with at least one compound of the formula (V)

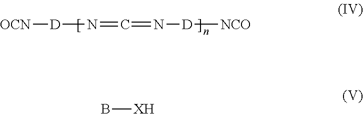

where n, D, B and X have the definitions already given.

This reaction is preferably conducted at a temperature in the range from 20 to 140° C., especially 30 to 120° C., preferably 40 to 100° C., optionally in the presence of at least one catalyst.

Preference is given to conducting the reaction in the absence of solvents.

Preference is given to choosing a stoichiometric or slightly superstoichiometric ratio between the HX groups of the compound of the formula (V) and the isocyanate groups of the carbodiimide of the formula (IV), and to conducting the reaction in such a way that all the isocyanate groups are converted. Preference is given to using the resultant carbodiimide adduct of the formula (II) without further processing or purification.

Suitable compounds of the formula (V) are especially mercaptans, alcohols or primary or secondary amines.

Particularly suitable compounds of the formula (V) are polyether monools having an average molecular weight in the range from 150 to 2′000 g/mol, preferably 200 to 1′000 g/mol, especially 2-(ω-(alkyloxy)poly(oxyethylene))ethylol, 2-(ω-(alkyloxy)poly-(oxyethyleneoxypropylene))ethylol, 2-(ω-(alkyloxy)poly(oxyethyleneoxypropylene))-propylol or 2-(ω-(alkyloxy)poly(oxypropylene))propylol, where alkyl represents methyl, ethyl, butyl, 2-ethylhexyl, lauryl, cocoalkyl or oleyl. Such compounds of the formula (V) enable carbodiimide adducts that are liquid at room temperature and have readily manageable viscosity.

Suitable carbodiimides of the formula (IV) are reaction products of at least one diisocyanate of the formula D-(NCO)₂ in the presence of a carbodiimidization catalyst while heating and with release of CO₂. Preferred carbodiimidization catalysts are phosphylene oxides, especially 3-methyl-1-phenyl-2-phosphylene 1-oxide or 1-methyl-2(3)-phosphylene 1-oxide.

Suitable diisocyanates of the formula D-(NCO)₂ are especially 2-methylpentamethylene 1,5-diisocyanate, hexamethylene 1,6-diisocyanate (HDI), 2,2(4),4-trimethyl-hexamethylene 1,6-diisocyanate (TMDI), 1-isocyanato-3,3,5-trimethyl-5-isocyanatomethylcyclohexane (IPDI), 1,3-bis-(isocyanatomethyl)cyclohexane, 1,4-bis(isocyanatomethyl)cyclohexane, m-xylylene diisocyanate (m-XDI), or perhydro-diphenylmethane 4,4′-diisocyanate (H₁₂MDI), especially HDI or IPDI or H₁₂MDI.

Preference is given to conducting the process for preparing the guanidine of the formula (I) in that
first at least one diisocyanate of the formula D-(NCO)₂ is converted in the presence of a carbodiimidization catalyst while heating and with release of CO₂,
then the resultant carbodiimide of the formula (IV) is reacted with at least one compound of the formula (V),
and finally the resultant carbodiimide adduct of the formula (II) is reacted with at least one amine of the formula (III),

as described above in each case.

The reaction product from this process is preferably used without workup or purification.

The described process for preparing the guanidine of the formula (I) is performable in a surprisingly quick and simple manner, especially without requiring the use of auxiliaries or processing or purification of the reaction product, and proceeds from readily available, inexpensive starting materials. It can be conducted as a one-pot reaction or in multiple stages in which the carbodiimide of the formula (IV) and/or the carbodiimide adduct of the formula (II) is isolated.

Suitable carbodiimide adducts of the formula (II) are also commercially available, for example Picassian® XL-725 (from Stahl Polymers).

The invention further provides for the use of the guanidine of the formula (I) as catalyst for the crosslinking of a functional compound. The guanidine of the formula (I) here catalyzes the crosslinking reaction of the reactive groups or the curing of the functional compound and curable compositions thereof.

Preferred reactive groups of the functional compound are silane groups, isocyanate groups, epoxy groups or cyanate ester groups.

Suitable functional compounds are especially
silanes,
polymers having silane groups,
polyisocyanates,
polymers having isocyanate groups, especially polyurethane polymers having isocyanate groups,
compounds having glycidoxy groups, especially epoxy resins,
cyanate ester resins, or
polymers having various reactive groups among those specified, especially compounds or polymers having isocyanate and silane groups or having isocyanate and epoxy groups.

The functional compound is preferably a silane, a polymer having silane groups, a polyisocyanate or a polyurethane polymer having isocyanate groups.

Suitable polyisocyanates are especially monomeric diisocyanates, or oligomers or polymers or derivatives of monomeric diisocyanates, or any mixtures thereof.

Suitable monomeric diisocyanates are especially tolylene 2,4- or 2,6-diisocyanate or any mixtures of these isomers (TDI), diphenylmethane 4,4′-, 2,4′- or 2,2′-diisocyanate or any mixtures of these isomers (MDI), mixtures of MDI and MDI homologs (polymeric MDI or PMDI), phenylene 1,3- or 1,4-diisocyanate, 2,3,5,6-tetramethyl-1,4-diisocyanatobenzene, naphthalene 1,5-diisocyanate (NDI), 3,3′-dimethyl-4,4′-diisocyanatodiphenyl (TODI), dianisidine diisocyanate (DADI), tetramethylene 1,4-diisocyanate, 2-methylpentamethylene 1,5-diisocyanate, hexamethylene 1,6-diisocyanate (HDI), 2,2(4),4-trimethylhexamethylene 1,6-diisocyanate (TMDI), decamethylene 1,10-diisocyanate, dodecamethylene 1,12-diisocyanate, lysine or lysine ester diisocyanate, cyclohexane 1,3- or 1,4-diisocyanate, 1-methyl-2,4- or -2,6-diisocyanatocyclohexane or any desired mixtures of these isomers (HTDI or H₆TDI), 1-isocyanato-3,3,5-trimethyl-5-isocyanatomethylcyclohexane (isophorone diisocyanate or IPDI), perhydro(diphenylmethane 2,4′- or 4,4′-diisocyanate) (HMDI or H₁₂MDI), 1,4-diisocyanato-2,2,6-trimethylcyclohexane (TMCDI), 1,3- or 1,4-bis(isocyanatomethyl)cyclohexane, m- or p-xylylene diisocyanate (m- or p-XDI), m- or p-tetramethylxylylene 1,3- or 1,4-diisocyanate (m- or p-TMXDI) or bis(1-isocyanato-1-methylethyl)naphthalene.

Among these, preference is given to MDI, TDI, IPDI or HDI.

Suitable oligomers, polymers or derivatives of monomeric diisocyanates are especially derived from MDI, TDI, HDI or IPDI.

Particularly preferred polyisocyanates are forms of MDI that are liquid at room temperature and have a high content of diphenylmethane 4,4′-diisocyanate. What is called “liquid MDI” is especially either diphenylmethane 4,4′-diisocyanate liquefied by partial chemical modification—especially carbodiimidization or uretonimine formation—or a mixture of diphenylmethane 4,4′-diisocyanate with other MDI isomers (2,4′-diphenylmethane diisocyanate and/or 2,2′-diphenylmethane diisocyanate) or MDI oligomers or MDI homologs that has been brought about selectively by blending or results from the production process.

Suitable polyurethane polymers having isocyanate groups are especially obtained from the reaction of at least one polyol with a superstoichiometric amount of at least one polyisocyanate, especially a diisocyanate. The reaction is preferably conducted with exclusion of moisture at a temperature in the range from 50 to 160° C., optionally in the presence of suitable catalysts. The excess of polyisocyanate is preferably chosen so as to leave, in the polyurethane polymer after the conversion of all hydroxyl groups, a content of free isocyanate groups in the range from 1% to 30% by weight, preferably 1.5% to 25% by weight, more preferably 2% to 20% by weight. The polyurethane polymer can optionally be prepared with additional use of plasticizers or solvents, in which case the plasticizers or solvents used do not contain any groups reactive toward isocyanates.

Diisocyanates suitable for this purpose are especially MDI, TDI, PMDI, HDI, IPDI, H₁₂MDI, or oligomers or derivatives of these diisocyanates.

Polyols suitable for this purpose are especially polyether polyols, preferably polyoxyalkylene polyols, which are polymerization products of ethylene oxide or 1,2-propylene oxide or 1,2- or 2,3-butylene oxide or oxetane or tetrahydrofuran or mixtures thereof, possibly polymerized with the aid of a starter molecule having two or more active hydrogen atoms; polyester polyols, preferably products from the polycondensation of diols or triols with lactones or dicarboxylic acids or esters or anhydrides thereof; polycarbonate polyols; OH-terminal block copolymers having at least two different blocks having polyether, polyester or polycarbonate units; polyacrylate polyols or polymethacrylate polyols; polyhydroxy-functional fats or oils, especially natural fats or oils; or polyhydrocarbon polyols, for example polyhydroxy-functional polyolefins, especially polybutadienepolyols.

Also especially suitable are mixtures of the polyols mentioned.

Especially suitable are diols or triols or mixtures thereof.

The polyurethane polymer having isocyanate groups preferably has an average molecular weight in the range from 350 to 30′000 g/mol, especially 1′000 to 15′000 g/mol.

Particularly preferred functional compounds are silanes and or polymers having silane groups.

The guanidine of the formula (I) has a strong catalytic effect on the hydrolysis and condensation reactions of silane groups. Silanes and polymers having silane groups therefore cure rapidly even with a relatively small amount of this catalyst.

Suitable silanes are especially tetramethoxysilane, tetraethoxysilane, methyltrimethoxysilane, ethyltrimethoxysilane, propyltrimethoxysilane, octyltrimethoxysilane, isooctyltrimethoxysilane, vinyltrimethoxysilane, phenyltrimethoxysilane, methyltriethoxysilane, octyltriethoxysilane, isooctyltriethoxysilane, vinyltriethoxysilane, phenyltriethoxysilane, aminosilanes such as, in particular, 3-aminopropyltrimethoxysilane, 3-aminopropyldiethoxymethylsilane, N-(2-aminoethyl)-3-aminopropyltrimethoxysilane, N-(2-aminoethyl)-3-aminopropyldiethoxymethylsilane, N-(2-aminoethyl)-N′-[3-(trimethoxysilyl)propyl]ethylenediamine or analogs thereof with ethoxy rather than methoxy groups, or N-phenyl-, N-cyclohexyl- or N-alkylaminosilanes, mercaptosilanes, epoxysilanes such as, in particular, 3-glycidoxypropyltrimethoxysilane or 3-glycidoxypropyltriethoxysilane, or 3-ureidopropyltrimethoxysilane, (meth)acryloylsilanes, anhydridosilanes, carbamatosilanes, or iminosilanes, adducts of primary aminosilanes with epoxysilanes or (meth)acryloylsilanes or anhydridosilanes, or oligomeric forms of these silanes.

A suitable polymer having silane groups is especially a polyorganosiloxane having terminal silane groups or an organic polymer having silane groups.

A preferred polyorganosiloxane having terminal silane groups has the formula (VI)

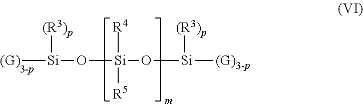

where

R³, R⁴ and R⁵ are each independently a monovalent hydrocarbyl radical having 1 to 12 carbon atoms,

G is a hydroxyl radical or an alkoxy, acetoxy, ketoximato, amido or enoxy radical having 1 to 13 carbon atoms,

p is 0, 1 or 2, and

m is an integer in the range from 50 to about 2′500.

R³ is preferably methyl, vinyl or phenyl.

R⁴ and R⁵ are preferably each independently an alkyl radical having 1 to 5, preferably 1 to 3, carbon atoms, especially methyl.

G is preferably a hydroxyl radical or an alkoxy or ketoximato radical having 1 to 6 carbon atoms, especially a hydroxyl, methoxy, ethoxy, methylethylketoximato or methylisobutylketoximato radical.

More preferably, G is a hydroxyl radical.

p is preferably 0 or 1, especially 0.

In addition, m is preferably selected such that the polyorganosiloxane of the formula (VI) has a viscosity at room temperature in the range from 100 to 500′000 mPa·s, especially from 1′000 to 100′000 mPa·s.

Suitable commercially available polyorganosiloxanes of the formula (VI) are available, for example, from Wacker, Momentive Performance Materials, GE Advanced Materials, Dow Corning, Bluestar Silicones or Shin-Etsu.

A suitable organic polymer having silane groups is especially a polyolefin, polyether, polyester, polyamide, poly(meth)acrylate or mixed forms of these polymers, each bearing one or preferably more than one silane group. The silane groups may be in pendant positions in the chain or in terminal positions and are bonded to the organic polymer via a carbon atom.

More preferably, the organic polymer having silane groups is a polyolefin having silane groups or a polyether having silane groups or a polyester having silane groups or a poly(meth)acrylate having silane groups or a mixed form of these polymers.

Most preferably, the organic polymer having silane groups is a polyether having silane groups.

The silane groups present in the organic polymer having silane groups are preferably alkoxysilane groups, especially alkoxysilane groups of the formula (VII)

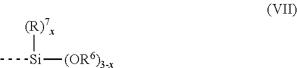

where

R⁶ is a linear or branched monovalent hydrocarbyl radical having 1 to 5 carbon atoms, especially methyl, ethyl or isopropyl,

R⁷ is a linear or branched monovalent hydrocarbyl radical having 1 to 8 carbon atoms, especially methyl, and

x has a value of 0 or 1 or 2, preferably 0 or 1, especially 0.

More preferably, R⁶ is methyl or ethyl.

Particular preference is given to trimethoxysilane groups, dimethoxymethylsilane groups or triethoxysilane groups.

Methoxysilane groups have the advantage here that they are particularly reactive and crosslink rapidly, and ethoxysilane groups have the advantage that they are particularly storage-stable and release comparatively nontoxic ethanol in the course of crosslinking.

The organic polymer having silane groups has an average of preferably 1.3 to 4, especially 1.5 to 3, more preferably 1.7 to 2.8, silane groups per molecule.

The silane groups are preferably terminal.

The organic polymer having silane groups preferably has an average molecular weight in the range from 1′000 to 30′000 g/mol, especially from 2′000 to 20′000 g/mol.

The organic polymer having silane groups preferably has a silane equivalent weight of 300 to 25′000 g/eq, especially 500 to 15′000 g/eq. The organic polymer having silane groups may be solid or liquid at room temperature. It is preferably liquid at room temperature.

Most preferably, the organic polymer having silane groups is a polyether having silane groups which is liquid at room temperature, where the silane groups are especially dialkoxysilane groups and/or trialkoxysilane groups, more preferably trimethoxysilane groups or triethoxysilane groups.

Processes for preparing polyethers having silane groups are known to the person skilled in the art.

In a preferred process, polyethers having silane groups are obtainable from the reaction of polyethers containing allyl groups with hydrosilanes, optionally with chain extension using diisocyanates for example.

In a further preferred process, polyethers having silane groups are obtainable from the copolymerization of alkylene oxides and epoxysilanes, optionally with chain extension using diisocyanates for example.

In a further preferred process, polyethers having silane groups are obtainable from the reaction of polyether polyols with isocyanatosilanes, optionally with chain extension using diisocyanates.

In a further preferred process, polyethers having silane groups are obtainable from the reaction of polyethers having isocyanate groups, especially NCO-terminated urethane polyethers from the reaction of polyether polyols with a superstoichiometric amount of polyisocyanates, or with aminosilanes, hydroxysilanes or mercaptosilanes. Polyethers having silane groups from this process are particularly preferred. This process enables the use of a multiplicity of commercially readily available inexpensive starting materials by means of which different polymer properties are obtainable, for example high extensibility, high strength, low modulus of elasticity, low glass transition temperature or high weathering resistance.

It is particularly preferable when the polyether having silane groups is obtainable from the reaction of NCO-terminated urethane polyethers with aminosilanes or hydroxysilanes. Suitable NCO-terminated urethane polyethers are obtainable from the reaction of polyether polyols, especially polyoxyalkylene diols or polyoxyalkylene triols, preferably polyoxypropylene diols or polyoxypropylene triols, with a superstoichiometric amount of polyisocyanates, especially diisocyanates.

It is preferable when the reaction between the polyisocyanate and the polyether polyol is conducted with exclusion of moisture at a temperature of 50° C. to 160° C., optionally in the presence of suitable catalysts, wherein the polyisocyanate has been dosed such that the isocyanate groups thereof are present in a stoichiometric excess in relation to the hydroxyl groups of the polyol. In particular, the excess of polyisocyanate is chosen such that in the resulting urethane polyether, after the reaction of all hydroxyl groups, there remains a content of free isocyanate groups of 0.1% to 5% by weight, preferably 0.2% to 4% by weight, more preferably 0.3% to 3% by weight.

Preferred diisocyanates are selected from the group consisting of HDI, IPDI, TDI and MDI. Particular preference is given to IPDI or TDI. Most preferred is IPDI. In this way, polyethers having silane groups with particularly good lightfastness are obtained.

Especially suitable as polyether polyols are polyoxyalkylenediols or polyoxyalkylenetriols having a degree of unsaturation lower than 0.02 meq/g, especially lower than 0.01 meq/g, and an average molecular weight in the range from 400 to 25′000 g/mol, especially 1′000 to 20′000 g/mol.

In addition to polyether polyols it is also possible to use proportions of other polyols, especially polyacrylate polyols and low molecular weight diols or triols.

Suitable aminosilanes for the reaction with an NCO-terminated urethane polyether are primary and secondary aminosilanes. Preference is given to 3-aminopropyltrimethoxysilane, 3-aminopropyldimethoxymethylsilane, 4-aminobutyltrimethoxysilane, 4-amino-3-methylbutyltrimethoxysilane, 4-amino-3,3-dimethylbutyltrimethoxysilane, N-butyl-3-aminopropyltrimethoxysilane, N-phenyl-3-aminopropyltrimethoxysilane, adducts formed from primary aminosilanes such as 3-aminopropyltrimethoxysilane, 3-aminopropyldiethoxymethylsilane or N-(2-aminoethyl)-3-aminopropyltrimethoxysilane and Michael acceptors such as acrylonitrile, (meth)acrylic esters, (meth)acrylamides, maleic or fumaric diesters, citraconic diesters or itaconic diesters, especially dimethyl or diethyl N-(3-trimethoxysilylpropyl)aminosuccinate. Likewise suitable are analogs of the recited aminosilanes with ethoxy or isopropoxy groups in place of the methoxy groups on the silicon.

Suitable hydroxysilanes for the reaction with an NCO-terminated urethane polyether are especially obtainable from the addition of aminosilanes onto lactones or onto cyclic carbonates or onto lactides.

Aminosilanes suitable for this purpose are especially 3-aminopropyltrimethoxysilane, 3-aminopropyltriethoxysilane, 4-aminobutyltrimethoxysilane, 4-aminobutyltriethoxysilane, 4-amino-3-methylbutyltrimethoxysilane, 4-amino-3-methylbutyltriethoxysilane, 4-amino-3,3-dimethylbutyltrimethoxysilane, 4-amino-3,3-dimethylbutyltriethoxysilane, 2-aminoethyltrimethoxysilane or 2-aminoethyltriethoxysilane. Particular preference is given to 3-aminopropyltrimethoxysilane, 3-aminopropyltriethoxysilane, 4-amino-3,3-dimethylbutyltrimethoxysilane or 4-amino-3,3-dimethylbutyltriethoxysilane.

Suitable lactones are especially γ-valerolactone, γ-octalactone, δ-decalactone, and ε-decalactone, especially γ-valerolactone.

Suitable cyclic carbonates are especially 4,5-dimethyl-1,3-dioxolan-2-one, 4,4-dimethyl-1,3-dioxolan-2-one, 4-ethyl-1,3-dioxolan-2-one, 4-methyl-1,3-dioxolan-2-one or 4-(phenoxymethyl)-1,3-dioxolan-2-one.

Suitable lactides are especially 1,4-dioxane-2,5-dione (lactide formed from 2-hydroxyacetic acid, also called “glycolide”), 3,6-dimethyl-1,4-dioxane-2,5-dione (lactide formed from lactic acid, also called “lactide”) or 3,6-diphenyl-1,4-dioxane-2,5-dione (lactide formed from mandelic acid).

Preferred hydroxysilanes that are obtained in this way are N-(3-triethoxysilylpropyl)-2-hydroxypropanamide, N-(3-triethoxysilylpropyl)-4-hydroxypentanamide, N-(3-triethoxysilylpropyl)-4-hydroxyoctanamide, N-(3-triethoxysilylpropyl)-5-hydroxydecanamide, N-(3-triethoxysilylpropyl)-2-hydroxypropyl carbamate and the corresponding silanes with methoxy in place of the ethoxy groups.

Suitable hydroxysilanes are additionally also obtainable from the addition of aminosilanes onto epoxides or from the addition of amines onto epoxysilanes. Preferred hydroxysilanes which are obtained in this way are 2-morpholino-4(5)-(2-trimethoxysilylethyl)cyclohexan-1-ol, 2-morpholino-4(5)-(2-triethoxysilyl-ethyl)cyclohexan-1-ol or 1-morpholino-3-(3-(triethoxysilyl)propoxy)propan-2-ol.

Further suitable polyethers having silane groups are commercially available products, especially the following: MS Polymer™ (from Kaneka Corp.; especially the S203H, S303H, S227, S810, MA903 and S943 products); MS Polymer™ or Silyl™ (from Kaneka Corp.; especially the SAT010, SAT030, SAT200, SAX350, SAX400, SAX725, MAX450, MAX951 products); Excestar® (from Asahi Glass Co. Ltd.; especially the S2410, S2420, S3430, S3630 products); SPUR+* (from Momentive Performance Materials; especially the 1010LM, 1015LM, 1050MM products); Vorasil™ (from Dow Chemical Co.; especially the 602 and 604 products); Desmoseal® (from Covestro AG; especially the S XP 2458, S XP 2636, S XP 2749, S XP 2774 and S XP 2821 products), TEGOPAC® (from Evonik Industries AG; especially the Seal 100, Bond 150, Bond 250 products), Polymer HBZ (from Hanse Chemie AG/Evonik Industries AG, especially the 47, 48, 61, 61LV, 77, 80, 81 products); Geniosil® STP (from Wacker Chemie AG; especially the E10, E15, E30, E35 products).

Particularly preferred organic polymers having silane groups have end groups of the formula (VIII)

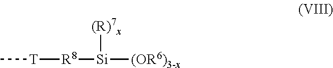

where

R⁸ is a divalent linear or branched hydrocarbyl radical which has 1 to 12 carbon atoms and optionally has cyclic and/or aromatic moieties and optionally one or more heteroatoms, especially one or more nitrogen atoms, T is a divalent radical selected from —O—, —S—, —N(R⁹)—, —O—CO—N(R⁹)—, —N(R⁹)—CO—O— and —N(R⁹)—CO—N(R⁹)—,
where R⁹ is a hydrogen radical or a linear or branched hydrocarbyl radical having 1 to 20 carbon atoms which optionally has cyclic moieties and which optionally has an alkoxysilane, ether or carboxylic ester group, and

R⁶, R⁷ and x are as defined above.

It is preferable when R⁸ is 1,3-propylene or 1,4-butylene, wherein butylene may be substituted by one or two methyl groups.

It is particularly preferable when R⁸ is 1,3-propylene.

Particularly preferred functional compounds are polyethers having silane groups.

The invention further provides a curable composition comprising at least one guanidine of the formula (I) as described above. In this case, the guanidine of the formula (I) catalyzes the crosslinking or curing of the composition.

Preferably, the curable composition contains reactive groups selected from silane groups, isocyanate groups, epoxy groups and cyanate ester groups.

More preferably, the curable composition contains silane groups and/or isocyanate groups, especially silane groups.

Preferably, the curable composition comprises at least one polyisocyanate or at least one polyurethane polymer having isocyanate groups or at least one silane or at least one polymer having silane groups, as described above.

More preferably, the curable composition comprises at least one silane and/or at least one polymer having silane groups.

Preferably, the curable composition is used for bonding, sealing, insulating, coating or pretreating in construction and industrial applications, especially as concrete element adhesive, facade adhesive, parquet adhesive, window profile adhesive, anchoring adhesive, assembly adhesive, bodywork adhesive, pane adhesive, sandwich element adhesive, lining adhesive, laminate adhesive, packaging adhesive, joint sealant, floor grout, spackling compound, sealing membrane, weld or crimp seam sealant, cavity seal, building foam, furniture foam, filter foam, insulation foam, sound insulation foam, packaging foam, bodywork foam, floor covering, floor coating, balcony coating, roof coating, concrete protection coating, parking garage coating, pipe coating, anticorrosion coating, textile coating, primer, activator or primer coat, or as molding, semifinished product, film or fiber, especially as cushioning, pillow, mattress, shoe sole, shock absorber, damping element, gasket, tire, roll, bearing, drum, conveyor belt, hose, housing, window profile, insulation panel, model construction panel, sandwich element, fiber composite body, implant, packaging film, lamination film or textile fiber.

In particular, the curable composition is an adhesive or a sealant or a coating. In one embodiment of the invention, the curable composition is especially a pretreatment agent, especially an adhesion promoter solution, an activator or a primer. In this case, the composition, as well as the guanidine of the formula (I), especially comprises at least one silane and at least one solvent, and optionally further constituents such as, in particular, titanates and/or zirconates, further catalysts and optionally fillers, wetting agents, polyisocyanates, polyurethane polymers having isocyanate and/or silane groups, or epoxy resins.

Such a pretreatment agent is especially used for pretreatment of substrates which are subsequently contacted after a suitable flash-off time with a further curable composition, especially a coating, an adhesive or a sealant, and the pretreatment agent especially improves the adhesion of the coating or the adhesive or sealant.

Most preferably, the curable composition comprises at least one polymer having silane groups, especially an organic polymer having silane groups, as described above.

A composition of this kind has good storage stability with no catalyst-induced propensity to separation, and because of the low toxicity and low volatility of the guanidine of the formula (I) allows a low hazard classification and enables low-emissions and largely odorless products that cure rapidly and at the same time form a mechanically high-quality and durable material. A particularly advantageous circumstance here is that this material shows barely any propensity to migration-related defects such as exudation or substrate soiling, by contrast with compositions comprising catalysts according to the prior art, for example DBU or TMG. Compositions comprising such catalysts known from the prior art have a propensity to migration effects, which can be manifested prior to curing by separation and after curing by tacky and/or greasy surfaces and/or substrate soiling. Particularly the latter effects are extremely undesirable, since tacky and greasy surfaces are rapidly soiled and are difficult to paint over, and substrate contaminants can lead to lasting discoloration.

Preferably, the guanidine of the formula (I) is present in the curable composition in such an amount that the concentration of guanidine groups based on the amount of the functional compound, especially based on the amount of the polymer having silane groups, is in the range from 0.1 to 50 mmol/100 g, preferably 0.2 to 50 mmol/100 g, especially 0.5 to mmol/100 g.

Such a composition has good storability and rapid curing.

In addition to the guanidine of the formula (I), the composition may comprise further catalysts which especially catalyze the crosslinking of isocyanate groups and/or silane groups. Suitable further catalysts are especially metal compounds and/or basic nitrogen or phosphorus compounds.

Suitable metal compounds are especially compounds of tin, titanium, zirconium, aluminum or zinc, especially diorganotin(IV) compounds such as in particular dibutyltin(IV) diacetate, dibutyltin(IV) dilaurate, dibutyltin(IV) dineodecanoate or dibutyltin(IV) bis(acetylacetonate) and dioctyltin(IV) dilaurate and also titanium(IV) or zirconium(IV) or aluminum(III) or zinc(II) complexes having in particular alkoxy, carboxylate, 1,3-diketonate, 1,3-ketoesterate or 1,3-ketoamidate ligands.

Suitable basic nitrogen or phosphorus compounds are especially imidazoles, pyridines, phosphazene bases or preferably amines, hexahydrotriazines, biguanides, amidines or further guanidines.

Suitable amines are, in particular, alkyl-, cycloalkyl- or aralkylamines such as triethylamine, triisopropylamine, 1-butylamine, 2-butylamine, tert-butylamine, 3-methyl-1-butylamine, 3-methyl-2-butylamine, dibutylamine, tributylamine, hexylamine, dihexylamine, cyclohexylamine, dicyclohexylamine, dimethylcyclohexylamine, benzylamine, dibenzylamine, dimethylbenzylamine, octylamine, 2-ethylhexylamine, di-(2-ethylhexyl)amine, laurylamine, N,N-dimethyllaurylamine, stearylamine, N,N-dimethylstearylamine; fatty amines derived from natural fatty acid mixtures; aliphatic, cycloaliphatic or araliphatic diamines such as ethylenediamine, butanediamine, hexamethylenediamine, dodecanediamine, neopentanediamine, 2-methylpentamethylenediamine (MPMD), 2,2(4),4-trimethylhexamethylenediamine (TMD), isophoronediamine (IPD), 2,5(2,6)-bis(aminomethyl)bicyclo[2.2.1]heptane (NBDA), xylylene-1,3-diamine (MXDA), N,N′-di(tert-butyl)ethylenediamine, N,N,N′,N′-tetramethylethylenediamine, N,N,N′,N′-tetramethylpropylenediamine, N,N,N′,N′-tetramethylhexamethylenediamine, 3-(dimethylamino)propylamine, 3-(methylamino)propylamine, 3-(cydohexylamino)propylamine, piperazine, N-methylpiperazine, N,N′-dimethylpiperazine, 1,4-diazabicyclo[2.2.2]octane (DABCO), fatty polyamines such as N-cocoalkylpropane-1,3-diamine; polyalkyleneamines such as diethylenetriamine, dipropylenetriamine, triethylenetetramine (TETA), tetraethylenepentamine (TEPA), pentamethylenehexamine (PEHA), 3-(2-aminoethyl)aminopropylamine, N,N′-bis(3-aminopropyl)ethylenediamine, N-(3-aminopropyl)-N-methylpropanediamine, bis(3-dimethylaminopropyl)amine, N-(3-dimethylaminopropyl)propylene-1,3-diamine, N-(2-aminoethyl)piperazine (N-AEP), N-(2-aminopropyl)piperazine, N,N′-di-(2-aminoethyl)piperazine, 1-methyl-4-(2-dimethylaminoethyl)piperazine, N,N,N′,N″,N″-pentamethyldiethylenetriamine, N,N,N′,N″,N″-pentamethyldipropylenetriamine, polyethyleneimines obtainable for example under the trade names Lupasol® (from BASF) and Epomin® (from Nippon Shokubai); ether amines, such as, in particular, 2-methoxyethylamine, 2-ethoxyethylamine, 3-methoxypropylamine, 3-ethoxypropylamine, 3-(2-ethylhexyloxy)propylamine, 3-(2-methoxyethoxy)propylamine, morpholine, N-methylmorpholine, N-ethylmorpholine, 2-aminoethylmorpholine, bis(2-aminoethyl) ether, bis(dimethylaminoethyl) ether, bis(morpholinoethyl) ether (DMDEE), N,N,N′-trimethyl-N′-hydroxyethylbis(2-aminoethyl) ether, 3,6-dioxaoctane-1,8-diamine, 4,7-dioxadecane-1,10-diamine, 4,7-dioxadecane-2,9-diamine, 4,9-dioxadodecane-1,12-diamine, 5,8-dioxadodecane-3,10-diamine, 4,7,10-trioxatridecane-1,13-diamine, or 2-aminopropyl-terminated glycols, of the kind obtainable for example under the trade name Jeffamine® (from Huntsman); amino alcohols, such as, in particular, ethanolamine, isopropanolamine, diethanolamine, diisopropanolamine, triethanolamine, triisopropanolamine, N-butylethanolamine, diglycolamine, N,N-diethylethanolamine, N-methyldiethanolamine, N-methyldiisopropylamine, N,N,N′-trimethylaminoethylethanolamine, N-(3-dimethylaminopropyl)-N,N-diisopropanolamine, N,N-bis(3-dimethylaminopropyl)-N-isopropanolamine, 2-(2-dimethylaminoethoxy)ethanolamine, or adducts of mono- and polyamines with epoxides or diepoxides; amines containing phenol groups, such as, in particular, condensation products of phenols, aldehydes, and amines (called Mannich bases and phenalkamines) such as, in particular, 2-(dimethylaminomethyl)phenol, 2,4,6-tris(dimethylaminomethyl)phenol, or polymers of phenol, formaldehyde, and N,N-dimethylpropane-1,3-diamine, and also phenalkamines obtainable commercially under the brand names Cardolite® (from Cardolite), Aradur® (from Huntsman), and Beckopoxe® (from Cytec); polyamines containing amide groups, so-called polyamidoamines, of the kind available commercially, for example, under the brand names Versamid® (from Cognis), Aradur® (from Huntsman), Euretek® (from Huntsman) or Beckopox® (from Cytec); or aminosilanes, such as, in particular, 3-aminopropyltrimethoxysilane, 3-aminopropyldimethoxymethylsilane, N-(2-aminoethyl)-3-aminopropyltrimethoxysilane, N-(2-aminoethyl)-3-aminopropyl-methyldimethoxysilane, N-(2-aminoethyl)-N′-[3-(trimethoxysilyl)propyl]ethylenediamine or their analogs with ethoxy in place of the methoxy groups on the silicon atom.

Suitable hexahydrotriazines are especially 1,3,5-hexahydrotriazine, 1,3,5-trimethylhexahydrotriazine or 1,3,5-tris(3-(dimethylamino)propyl)hexahydrotriazine.

Suitable biguanides are especially biguanide, 1-butylbiguanide, 1,1-dimethylbiguanide, 1-butylbiguanide, 1-phenylbiguanide or 1-(ω-tolyl)biguanide (OTBG).

Suitable amidines are especially 1,8-diazabicyclo[5.4.0]undec-7-ene (DBU), 1,5-diazabicyclo[4.3.0]non-5-ene (DBN), 6-dibutylamino-1,8-diazabicyclo[5.4.0]undeo-7-ene, 6-dibutylamino-1,8-diazabicyclo[5.4.0]undec-7-ene, N,N′-di-n-hexylacetamidine (DHA), 2-methyl-1,4,5,6-tetrahydropyrimidine, 1,2-dimethyl-1,4,5,6-tetrahydropyrimidine, 2,5,5-trimethyl-1,4,5,6-tetrahydropyrimidine, N-(3-trimethoxysilylpropyl)-4,5-dihydroimidazole or N-(3-triethoxy-silylpropyl)-4,5-dihydroimidazole.

Suitable further guanidines are especially 1-butylguanidine, 1,1-dimethylguanidine, 1,3-dimethylguanidine, 1,1,3,3-tetramethylguanidine (TMG), 2-(3-(trimethoxysilyl)propyl)-1,3,3-tetramethylguanidine, 2-(3-(methyldimethoxysilyl)propyl)-1,1,3,3-tetramethylguanidine, 2-(3-(triethoxysilyl)propyl)-1,1,3,3-tetramethylguanidine, 1,5,7-triazabicyclo-[4.4.0]dec-5-ene (TBD), 7-methyl-1,5,7-triazabicyclo[4.4.0]dec-5-ene, 7-cyclohexyl-1,5,7-triazabicyclo[4.4.0]dec-5-ene, 1-phenylguanidine, 1-(ω-tolyl)guanidine (OTG), 1,3-diphenylguanidine, 1,3-di(ω-tolyl)guanidine or 2-guanidinobenzimidazole.

In addition, the composition may comprise, as cocatalyst, an acid, especially a carboxylic acid. Preference is given to aliphatic carboxylic acids such as formic acid, lauric acid, stearic acid, isostearic acid, oleic acid, 2-ethyl-2,5-dimethylcaproic acid, 2-ethylhexanoic acid, neodecanoic acid, fatty acid mixtures from the hydrolysis of natural fats and oils or di- and polycarboxylic acids, especially poly(meth)acrylic acids.

In a preferred embodiment, the composition is essentially free of organotin compounds. Organotin-free compositions are advantageous in terms of protection of health and protection of the environment. More particularly, the tin content of the curable composition is less than 0.1% by weight, especially less than 0.05% by weight.

In a further preferred embodiment, the composition comprises a combination of at least one guanidine of the formula (I) and at least one organotin compound, especially a diorganotin(IV) compound such as those mentioned above. Such a composition has a high curing rate even in the case of a low tin content, which is advantageous for toxicological and environmental reasons.

In one embodiment, the composition additionally comprises, as well as the guanidine of the formula (I), at least one organotitanate. A combination of the guanidine of the formula (I) and an organotitanate has particularly high catalytic activity. This enables rapid curing with a comparatively small use amount of organotitanate.

Suitable organotitanates are especially titanium(IV) complexes.

Preferred organotitanates are especially selected from
titanium(IV) complexes having two 1,3-diketonate ligands, especially 2,4-pentanedionate (=acetylacetonate), and two alkoxide ligands;
titanium(IV) complexes having two 1,3-ketoesterate ligands, especially ethylacetoacetate, and two alkoxide ligands;
titanium(IV) complexes having one or more aminoalkoxide ligands, especially triethanolamine or 2-((2-aminoethyl)amino)ethanol, and one or more alkoxide ligands;
titanium(IV) complexes having four alkoxide ligands (orthotitanates);
and more highly condensed organotitanates, especially oligomeric titanium(IV) tetrabutoxide, also referred to as polybutyl titanate;

where suitable alkoxide ligands are especially isobutoxy, n-butoxy, isopropoxy, ethoxy and 2-ethyhexoxy.

Especially suitable are the commercially available products Tyzor® AA, GBA, GBO, AA-75, AA-65, AA-105, DC, BEAT, BTP, TE, TnBT, KTM, TOT, TPT or IBAY (all from Dorf Ketal); Tytan PBT, TET, X85, TAA, ET, S2, S4 or S6 (all from Borica Company Ltd.) and Ken-React® KR® TTS, 7, 9QS, 12, 26S, 33DS, 38S, 39DS, 44, 134S, 138S, 133DS, 158FS or LICA® 44 (all from Kenrich Petrochemicals).

Very particularly suitable organotitanates are selected from bis(ethylaceto-acetato)diisobutoxytitanium(IV) (commercially available, for example, as Tyzor® IBAY from Dorf Ketal), bis(ethylacetoacetato)diisopropoxytitanium(IV) (commercially available, for example, as Tyzor® DC from Dorf Ketal), bis(acetylacetonato)diisopropoxytitanium(IV), bis(acetylacetonato)diisobutoxy-titanium(IV), tris(oxyethyl)amineisopropoxytitanium(IV), bis[tris(oxyethyl)-amine]diisopropoxytitanium(IV), bis(2-ethylhexane-1,3-dioxy)titanium(IV), tris[2-((2-aminoethyl)amino)ethoxy]ethoxytitanium(IV), bis(neopentyl(diallyl)oxy)-diethoxytitanium(IV), tetra(isopropoxy)titanate, tetra(n-butoxy)titanate, tetra(2-ethylhexyloxy)titanate and polybutyl titanate.

Most preferred are bis(ethylacetoacetato)diisobutoxytitanium(IV) or bis(ethylacetoacetato)diisopropoxytitanium(IV).

The curable composition preferably comprises at least one further constituent selected from the group consisting of fillers, plasticizers, rheology additives, desiccants, adhesion promoters and crosslinkers. More preferably, it comprises any combination of two or more of these constituents.

Suitable fillers are especially inorganic or organic fillers, especially natural, ground or precipitated calcium carbonates, optionally coated with fatty acids, especially stearic acid, baryte (heavy spar), talcs, quartz flours, quartz sand, dolomites, wollastonites, kaolins, calcined kaolins, mica (potassium aluminum silicate), molecular sieves, aluminum oxides, aluminum hydroxides, magnesium hydroxide, silicas including finely divided silicas from pyrolysis processes, industrially produced carbon blacks, graphite, metal powders such as aluminum, copper, iron, silver or steel, PVC powder or hollow spheres.

Suitable plasticizers are especially trialkylsilyl-terminated polydialkylsiloxanes, preferably trimethylsilyl-terminated polydimethylsiloxanes, especially having viscosities in the range from 10 to 1′000 mPa·s, or corresponding compounds in which some of the methyl groups have been replaced by other organic groups, especially phenyl, vinyl or trifluoropropyl groups, called reactive plasticizers, in the form of monofunctional polysiloxanes, i.e. those that are reactive at one end, carboxylic esters such as phthalates, especially dioctyl phthalate, bis(2-ethylhexyl) phthalate, bis(3-propylheptyl) phthalate, diisononyl phthalate or diisodecyl phthalate, diesters of ortho-cydohexane-dicarboxylic acid, especially diisononyl 1,2-cyclohexanedicarboxylate, adipates, especially dioctyl adipate, bis(2-ethylhexyl) adipate, azelates, especially bis(2-ethylhexyl) azelate, sebacates, especially bis(2-ethylhexyl) sebacate or diisononyl sebacate, polyols, especially polyoxyalkylene polyols or polyester polyols, glycol ethers, glycol esters, organic phosphoric or sulfonic esters, sulfonamides, polybutenes, or fatty acid methyl or ethyl esters derived from natural fats or oils, also called “biodiesel”, plasticizers containing siloxane groups being particularly suitable for polymers having silane groups in the form of polyorganosiloxanes.

Suitable rheology additives are especially thickeners, especially sheet silicates such as bentonites, derivatives of castor oil, hydrogenated castor oil, polyamides, polyurethanes, urea compounds, fumed silicas, cellulose ethers or hydrophobically modified polyoxyethylenes.

Suitable desiccants are especially tetraethoxysilane, vinyltrimethoxysilane, vinyltriethoxysilane or organoalkoxysilanes which have a functional group in the a position to the silane group, especially N-(methyldimethoxysilylmethyl)-O-methylcarbamate, (methacryloyloxymethyl)silanes, methoxymethylsilanes, orthoformic esters, calcium oxide, molecular sieves, highly reactive isocyanates such as p-tosyl isocyanate, monomeric diisocyanates or monooxazolidines such as Incozol® 2 (from Incorez), especially vinyltrimethoxysilane or vinyltriethoxysilane.

Suitable adhesion promoters and/or crosslinkers are especially aminosilanes such as, in particular, 3-aminopropyltrimethoxysilane, 3-aminopropyldimethoxymethylsilane, N-(2-aminoethyl)-3-aminopropyltrimethoxysilane, N-(2-aminoethyl)-3-aminopropyldimethoxymethylsilane, N-(2-aminoethyl)-N′-[3-(trimethoxysilyl)propyl]ethylenediamine or the analogs thereof with ethoxy in place of methoxy groups, and also N-phenyl-, N-cyclohexyl- or N-alkylaminosilanes, mercaptosilanes, epoxysilanes, (meth)acrylosilanes, anhydridosilanes, carbamatosilanes, alkylsilanes or iminosilanes, oligomeric forms of these silanes, adducts formed from primary aminosilanes with epoxysilanes or (meth)acrylosilanes or anhydridosilanes, amino-functional alkylsilsesquioxanes, especially amino-functional methylsilsesquioxane or amino-functional propylsilsesquioxane. Especially suitable are 3-aminopropyltrimethoxysilane, 3-aminopropyltriethoxysilane, N-(2-aminoethyl)-3-aminopropyltrimethoxysilane, N-(2-aminoethyl)-3-aminopropyltriethoxysilane, 3-glycidoxypropyltrimethoxysilane, 3-glycidoxypropyltriethoxysilane or 3-ureidopropyltrimethoxysilane, or oligomeric forms of these silanes.

In a preferred embodiment, the composition comprises at least one desiccant and at least one adhesion promoter and/or crosslinker.

In a preferred embodiment, the composition does not comprise any phthalates as plasticizers. Such compositions are toxicologically advantageous and in some cases have fewer problems with migration effects.

If the composition comprises a polyisocyanate and/or a polyurethane polymer having isocyanate groups, additionally preferably, at least one polyfunctional compound reactive toward isocyanate groups is present, such as, in particular,
one or more polyols, especially the polyols mentioned as being suitable for the preparation of a polyurethane polymer having isocyanate groups. Preference is given to polyether polyols, polyester polyols, polycarbonate polyols, poly(meth)acrylate polyols or polybutadiene polyols. Particular preference is given to polyether polyols, especially polyoxypropylene polyols and/or ethylene oxide-terminated polyoxypropylene polyols. Preference is given to polyols having an average molecular weight in the range from 400 to 10′000 g/mol, especially 500 to 6′000 g/mol. Preference is given to polyols having an average OH functionality in the range from 1.6 to 4, especially 1.8 to 3, more preferably 2.2 to 3. Likewise suitable are polyether polyols with polymer particles dispersed therein, especially those with styrene-acrylonitrile particles (SAN) or polyurea or polyhydrazodicarbonamide particles (PHD).
chain extenders, especially ethane-1,2-diol, propane-1,3-diol, 2-methylpropane-1,3-diol, butane-1,4-diol, pentane-1,5-diol, neopentyl glycol, hexane-1,6-diol, 3-methylpentane-1,5-diol, heptane-1,7-diol, octane-1,8-diol, cyclohexane-1,3-dimethanol, cyclohexane-1,4-dimethanol, diethylene glycol or triethylene glycol;
amino alcohols, especially 2-aminoethanol, 2-(2-aminoethoxy)ethanol or 3-aminomethyl-3,5,5-trimethylcyclohexanol or derivatives thereof that have ether, ester or urethane groups;
compounds having blocked amino groups, especially aldimines, ketimines, enamines, oxazolidines, imidazolidines or hexahydropyrimidines;
or polyamines.

The composition may contain further constituents, especially the following auxiliaries and admixtures:
pigments, especially titanium dioxide or iron oxides;
dyes;
stabilizers against oxidation, heat, light or UV radiation;
natural resins, fats or oils such as rosin, shellac, linseed oil, castor oil or soya oil;
non-reactive polymers such as, in particular, homo- or copolymers of unsaturated monomers, especially from the group comprising ethylene, propylene, butylene, isobutylene, isoprene, vinyl acetate or alkyl (meth)acrylates, especially polyethylenes (PE), polypropylenes (PP), polyisobutylenes, ethylene-vinyl acetate copolymers (EVA) or atactic poly-α-olefins (APAO);
flame-retardant substances, especially the already mentioned fillers aluminum hydroxide and magnesium hydroxide, or, in particular, organic phosphoric esters such as, in particular, triethyl phosphate, tricresyl phosphate, triphenyl phosphate, diphenyl cresyl phosphate, isodecyl diphenyl phosphate, tris(1,3-dichloro-2-propyl) phosphate, tris(2-chloroethyl) phosphate, tris(2-ethylhexyl) phosphate, tris(chloroisopropyl) phosphate, tris(chloropropyl) phosphate, isopropylated triphenyl phosphate, mono-, bis- or tris(isopropylphenyl) phosphates of different degrees of isopropylation, resorcinol bis(diphenyl phosphate), bisphenol A bis(diphenyl phosphate) or ammonium polyphosphates;
fibers, especially glass fibers, carbon fibers, metal fibers, ceramic fibers or polymer fibers such as polyamide fibers or polyethylene fibers;
surface-active substances, especially wetting agents, leveling agents, deaerating agents or defoamers;
solvents;
biocides, especially algicides, fungicides or substances that inhibit fungal growth; and other substances customarily used in curable compositions.

It may be advisable to subject certain constituents to chemical or physical drying before mixing them into the composition.

The composition is preferably produced and stored under exclusion of moisture. It is typically storage-stable with exclusion of moisture in a suitable package or arrangement, such as, in particular, a cartridge, a bottle, a canister, a pouch, a bucket, a hobbock or a vat.

The composition may be in the form of a one-component composition or in the form of a multi-component, especially two-component, composition.

In the present document, “one-component” refers to a composition in which all constituents of the composition are stored in a mixture in the same container and which is curable with moisture.

In the present document, “two-component” refers to a composition in which the constituents of the composition are present in two different components which are stored in separate containers. Only shortly before or during the application of the composition are the two components mixed with one another, whereupon the mixed composition cures, optionally under the action of moisture.

If the composition comprises a polyisocyanate and/or a polyurethane polymer having isocyanate groups, it is preferably a two-component composition. In this case, one component contains the polyisocyanate and/or the polyurethane polymer having isocyanate groups and the other component contains the guanidine of the formula (I) and additionally at least one polyfunctional compound reactive toward isocyanate groups.

If the composition comprises an organic polymer having silane groups, it is preferably a one-component composition.

Any second or optionally further components is/are mixed with the first component prior to or during application, especially by means of a static mixer or by means of a dynamic mixer.

The composition is especially applied at ambient temperature, preferably within a temperature range between 0° C. and 45° C., especially 5° C. to 35° C., and also cures under these conditions.

On application, the crosslinking reaction of the functional groups commences, if appropriate under the influence of moisture.

Isocyanate groups present react with hydroxyl groups, or primary or secondary amino groups, or under the influence of moisture with blocked amino groups.

Any further isocyanate groups present react with one another under the influence of moisture.

Silane groups present can condense with silanol groups present to afford siloxane groups (Si—O—Si groups). Silane groups present can also be hydrolyzed on contact with moisture to give silanol groups (Si—OH groups) and can form siloxane groups (Si—O—Si groups) through subsequent condensation reactions.

As a result of these reactions, the composition ultimately cures. The guanidine of the formula (I) accelerates this curing.

If water is required for the curing, this can either come from the air (atmospheric humidity), or else the composition can be contacted with a water-containing component, for example by painting, for example with a smoothing agent, or by spraying, or water or a water-containing component can be added to the composition on application, for example in the form of a water-containing or water-releasing liquid or paste. A paste is especially suitable if the composition itself is in the form of a paste.

In the case of curing by means of atmospheric humidity, the composition cures from the outside inward, at first forming a skin on the surface of the composition. The so-called skin time is a measure of the curing rate of the composition. The speed of curing is generally determined by various factors, for example the availability of water, the temperature, etc.

The composition is suitable for a multitude of uses, especially as a paint, varnish or primer, as a resin for production of fiber composites, as a rigid foam, flexible foam, molding, elastomer, fiber, film or membrane, as a potting compound, sealant, adhesive, covering, coating or paint for construction and industrial applications, for example as a seam seal, cavity seal, electrical insulation compound, spackling compound, joint sealant, weld or crimp seam sealant, assembly adhesive, bodywork adhesive, glazing adhesive, sandwich element adhesive, lining adhesive, laminate adhesive, packaging adhesive, wood adhesive, parquet adhesive, anchoring adhesive, floor covering, floor coating, balcony coating, roof coating, concrete protection coating, parking garage coating, seal, pipe coating, anticorrosion coating, textile coating, damping element, sealing element or spackling compound.

The composition is particularly suitable as an adhesive and/or sealant, especially for joint sealing and for elastic adhesive bonds in construction and industrial applications, or as elastic coating with crack-bridging properties, especially for protection and/or sealing of, for example, roofs, floors, balconies, parking decks or concrete pipes.

The composition is thus preferably an adhesive or a sealant or a coating.

A composition of this kind typically comprises fillers, plasticizers, desiccants, adhesion promoters and/or crosslinkers and optionally further auxiliaries and additives.

For use as an adhesive or sealant the composition preferably has a pasty consistency with pseudoplastic properties. A pasty sealant or adhesive of this kind is especially applied to a substrate from standard cartridges which are operated manually, with compressed air or with a battery, or from a vat or hobbock via a delivery pump or an extruder, optionally via an application robot. For use as a coating the composition preferably has a liquid consistency at room temperature with self-leveling properties. It may be slightly thixotropic, such that the coating is applicable to inclined to vertical surfaces without flowing away immediately. It is especially applied by means of a roller or brush or by pouring-out and distribution by means, for example, of a roller, a scraper or a notched trowel.

During application the composition is preferably applied to at least one substrate.

Suitable substrates are especially
glass, glass ceramic, concrete, mortar, brick, tile, gypsum and natural rocks such as limestone, granite or marble;
metals and alloys such as aluminum, iron, steel or nonferrous metals, and also surface-finished metals or alloys such as galvanized or chromed metals;
leather, textiles, paper, wood, wood-based materials bonded with resins, for example phenolic, melamine or epoxy resins, resin-textile composites and further polymer composites;
plastics such as polyvinyl chloride (rigid and flexible PVC), acrylonitrile-butadiene-styrene copolymers (ABS), polycarbonate (PC), polyamide (PA), polyesters, poly(methyl methacrylate) (PMMA), epoxy resins, polyurethanes (PUR), polyoxymethylene (POM), polyolefins (PO), polyethylene (PE) or polypropylene (PP), ethylene/propylene copolymers (EPM) and ethylene/propylene/diene terpolymers (EPDM), and fiber-reinforced plastics such as carbon fiber-reinforced composite plastics (CFP), glass fiber-reinforced plastics (GFP) and sheet molding compounds (SMC), where the plastics may preferably have been surface-treated by means of plasma, corona or flames;
coated substrates such as powder-coated metals;
paints or varnishes, especially automotive topcoats, metal paints, furniture varnishes or wood varnishes.

If required, the substrates can be pretreated prior to the application of the composition, especially by physical and/or chemical cleaning methods or by the application of an adhesion promoter, an adhesion promoter solution or a primer.

The composition is particularly suitable for contact with substrates that are particularly sensitive to defects caused by migrating substances, especially by the formation of discoloration or specks. These are, in particular, fine-pore substrates such as marble, limestone or other natural stones, gypsum, cement mortar or concrete, but also plastics. Especially on PVC, severe discoloration is observed in the presence of catalysts, for example DBU or TMG, and cannot be removed by cleaning. No such effects are observed with the guanidine of the formula (I).

It is possible to bond or seal two identical or two different substrates, especially the aforementioned substrates.

After the curing of the composition, a cured composition is obtained.

The invention thus further provides a cured composition obtained from the composition described after curing thereof.

The use of the composition affords an article which has in particular been bonded, sealed or coated with the composition. The article is especially a built structure, especially a structure built by structural engineering or civil engineering, an industrially manufactured item or a consumable item, especially a window, a domestic appliance or a means of transport such as in particular an automobile, a bus, a truck, a rail vehicle, a ship, an aircraft or a helicopter; or the article may be an installable component thereof.

The curable composition is storable and applicable in a pleasant manner due to low odor. After application, it builds up strength surprisingly quickly, giving rise to mechanically high-quality and stable materials. The composition does not have a tendency to migration-related defects such as separation, exudation or substrate soiling either before or after curing.

### EXAMPLES

Working examples are adduced hereinafter, which are intended to elucidate the invention described in detail. It will be appreciated that the invention is not restricted to these described working examples.

“Standard climatic conditions” refer to a temperature of 23±1° C. and a relative air humidity of 50±5%.

Infrared spectra (FT-IR) were measured on a Nicolet iS5 FT-IR instrument from Thermo Scientific equipped with a horizontal ATR measurement unit with a diamond crystal. Liquid samples were applied undiluted as films; solid samples were dissolved in CH₂Cl₂.

The skin time (HBZ) was determined by applying a few grams of the composition to cardboard in a film thickness of about 2 mm and measuring under standard climatic conditions the time until, upon gentle tapping of the surface of the composition using an LDPE pipette, no residue remained on the pipette for the first time.

The characteristics of the surface were tested by touch.

The mechanical properties of tensile strength, elongation at break and modulus of elasticity (at 0-25% or at 0-5% and 0-50% elongation) were measured in accordance with DIN EN 53504 at a pulling speed of 200 mm/min. Viscosity was measured on a thermostated Rheotec RC30 cone-plate viscometer (cone diameter 50 mm, cone angle 1°, cone tip-plate distance 0.05 mm, shear rate 10 rpm).

### Carbodiimide Adducts of the Formula (II) Used:

### Carbodiimide Adduct A1:

Commercial carbodiimide adduct having a carbodiimide equivalent weight of about 700 g/mol (Picassian® XL-725 from Stahl Polymers).

### Carbodiimide Adduct A2:

In a round-bottom flask, 100.00 g of dicyclohexylmethane 4,4′-diisocyanate (Desmodur® W, from Covestro) and 0.07 g of 1-oxo-3-methyl-1-phenyl-2-phosphylene were mixed and heated under a nitrogen atmosphere. The mixture was stirred first at 180° C. for 2 h, then at 200° C. for 22 h, drawing vacuum (500 mbar) for 3 min every full hour. Thereafter, the content of free isocyanate groups determined by titrimetry was 11.4% by weight. 119.06 g of UCON® OSP-18 (polyalkylene glycol monool having an average molecular weight of 500 g/mol, from Dow) were added and the mixture was stirred at 140° C. After 16 h, no free isocyanate was detectable any longer by means of FT-IR spectroscopy. The polycarbodiimide adduct obtained was cooled down to room temperature and stored with exclusion of moisture. What was obtained was a yellowish, clear liquid having a calculated carbodiimide equivalent weight of 748 g/mol.

### Carbodiimide Adduct A3:

In a round-bottom flask, 50.00 g of dicyclohexylmethane 4,4′-diisocyanate (Desmodur® W, from Covestro), 50.00 g of hexamethylene 1,6-diisocyanate (Desmodur® H, from Covestro) and 0.08 g of 1-oxo-3-methyl-1-phenyl-2-phosphylene were mixed and heated under a nitrogen atmosphere. The mixture was stirred first at 180° C. for 2 h, then at 200° C. for 6 h, drawing vacuum (500 mbar) for 3 min every full hour. Thereafter, the content of free isocyanate groups determined by titrimetry was 14.5% by weight. 75.84 g of UCON® OSP-18 (polyalkylene glycol monool having an average molecular weight of 500 g/mol, from Dow) were added in order to convert half the isocyanate groups present and to lower the viscosity of the mixture. Ultimately, the mixture was stirred at 160° C. for a further 5 h, after which the content of free isocyanate groups was 4.3% by weight. A further 85.55 g of UCON® OSP-18 were added and the mixture was stirred at 140° C. After 18 h, no free isocyanate was detectable any longer by means of FT-IR spectroscopy. The polycarbodiimide adduct obtained was cooled down to room temperature and stored with exclusion of moisture. What was obtained was a yellowish, clear liquid of high viscosity having a calculated carbodiimide equivalent weight of 716 g/mol.

Preparation of guanidines of the formula (I):

### Guanidine G1:

In a round-bottom flask, 7.00 g of carbodiimide adduct A1 and 2.02 g of hexylamine were mixed under a nitrogen atmosphere and the mixture was stirred at 100° C. for 24 h. Then the carbodiimide band in the FT-IR at about 2120 cm⁻¹ had completely disappeared. 0.09 g of vinyltriethoxysilane was added and the mixture was cooled down to room temperature. What was obtained was a brownish, clear, odorless solid.

### Guanidine G2:

In a round-bottom flask, 7.00 g of carbodiimide adduct A1 and 1.42 g of 2-ethylhexylamine were mixed under a nitrogen atmosphere and the mixture was stirred at 80° C. for 24 h. Then the carbodiimide band in the FT-IR at about 2120 cm⁻¹ had completely disappeared. 0.09 g of vinyltriethoxysilane was added and the mixture was cooled down to room temperature. What was obtained was an odorless, brownish, clear liquid.

### Guanidine G3:

In a round-bottom flask, 10.00 g of carbodiimide adduct A2 and 1.90 g of 2-ethylhexylamine were mixed under a nitrogen atmosphere and the mixture was stirred at 120° C. for 48 h. Then the carbodiimide band in the FT-IR at about 2120 cm⁻¹ had completely disappeared. 0.12 g of vinyltriethoxysilane was added and the mixture was cooled down to room temperature. What was obtained was an odorless, yellow, clear liquid of high viscosity.

### Guanidine G4:

In a round-bottom flask, 10.00 g of carbodiimide adduct A3 and 1.97 g of 2-ethylhexylamine were mixed under a nitrogen atmosphere and the mixture was stirred at 120° C. for 48 h. Then the carbodiimide band in the FT-IR at about 2120 cm⁻¹ had completely disappeared. 0.10 g of vinyltriethoxysilane was added and the mixture was cooled down to room temperature. What was obtained was an odorless, yellow, clear liquid.

### Guanidine G5:

In a round-bottom flask, 7.00 g of carbodiimide adduct A1 and 2.02 g of 3-(2-ethylhexyloxy)propylamine were mixed under a nitrogen atmosphere and the mixture was stirred at 100° C. for 48 h. Then the carbodiimide band in the FT-IR at about 2120 cm⁻¹ had completely disappeared. What was obtained was a brownish, clear, odorless solid.

### Guanidine G6:

In a round-bottom flask, 10.00 g of carbodiimide adduct A2 and 2.77 g of 3-(2-ethylhexyloxy)propylamine were mixed under a nitrogen atmosphere and the mixture was stirred at 120° C. for 48 h. Then the carbodiimide band in the FT-IR at about 2120 cm⁻¹ had completely disappeared. What was obtained was an odorless, yellow, clear liquid.

### Guanidine G7:

In a round-bottom flask, 10.00 g of carbodiimide adduct A3 and 2.89 g of 3-(2-ethylhexyloxy)propylamine were mixed under a nitrogen atmosphere and the mixture was stirred at 120° C. for 48 h. Then the carbodiimide band in the FT-IR at about 2120 cm⁻¹ had completely disappeared. What was obtained was an odorless, yellow, clear liquid.

### Preparation of Polyethers Having Silane Groups:

### Polymer STP-1:

With exclusion of moisture, 1000 g of Acclaim® 12200 polyol (polyoxypropylenediol having a low level of unsaturation, from Covestro; OH number 11.0 mg KOH/g), 43.6 g of isophorone diisocyanate (IPDI; Vestanat® IPDI, from Evonik), 126.4 g of diisononyl cyclohexane-1,2-dicarboxylate (DINCH) and 0.1 g of bismuth tris(neodecanoate) (10% by weight in DINCH) were heated up to 90° C. while stirring constantly and left at this temperature until the content of free isocyanate groups determined by titrimetry had reached a stable value of 0.63% by weight. Subsequently, 63.0 g of diethyl N-(3-trimethoxysilylpropyl)aminosuccinate (adduct of 3-aminopropyltrimethoxysilane and diethyl maleate; prepared as per U.S. Pat. No. 5,364,955) were mixed in and the mixture was stirred at 90° C. until it was no longer possible to detect any free isocyanate by FT-IR spectroscopy. The polyether having trimethoxysilane groups obtained in this way was cooled down to room temperature and stored with exclusion of moisture.

### Polymer STP-2:

With exclusion of moisture, 1000 g of Acclaim® 12200 polyol (polyoxypropylenediol having a low level of unsaturation, from Covestro; OH number 11.0 mg KOH/g), 43.6 g of isophorone diisocyanate (IPDI; Vestanat® IPDI, from Evonik), 126.4 g of diisononyl cyclohexane-1,2-dicarboxylate (DINCH) and 0.1 g of bismuth tris(neodecanoate) (10% by weight in DINCH) were heated up to 90° C. while stirring constantly and left at this temperature until the content of free isocyanate groups determined by titrimetry had reached a stable value of 0.64% by weight. Subsequently, 70.6 g of diethyl N-(3-triethoxysilylpropyl)-aminosuccinate (adduct formed from 3-aminopropyltriethoxysilane and diethyl maleate) were mixed in and the mixture was stirred at 90° C. until it was no longer possible to detect any free isocyanate by means of FT-IR spectroscopy. The polyether having triethoxysilane groups obtained in this way was cooled down to room temperature and stored with exclusion of moisture.

### Commercial Catalysts Used:

DBU 1,8-diazabicyclo[5.4.0]undec-7-ene (Lupragen® N 700, from BASF) TMG 1,1,3,3-tetramethylguanidine (from Sigma-Aldrich)

### Compositions Based on Polymers Having Silane Groups:

Comparative examples in tables 1 to 4 are indicated by “(Ref)”.

### Compositions Z1 to Z9:

A composition composed of 97.6 g of polymer STP-1, 2.0 g of vinyltrimethoxysilane and 0.4 g of 3-aminopropyltrimethoxysilane was blended with various catalysts in the amount specified according to table 1, and the mixture was tested for viscosity at 25° C. and skin time (HBZ) under standard climatic conditions, before and after storage. Skin time serves as a measure of the activity of the catalyst in relation to the crosslinking reaction of the silane groups, i.e. of the crosslinking rate; the change in viscosity and the skin time after storage are a measure of storage stability of the composition. In addition, the mixture applied, after 24 h under standard climatic conditions, was tested as to whether the surface was dry as desired or whether a greasy film had formed, which is a sign of the exudation of the catalyst owing to poor compatibility with the cured polymer, and/or whether the surface was tacky, which is a sign of incomplete curing. In addition, the mixture was used to produce a film of thickness 2 mm, which was cured under standard climatic conditions for 7 days and tested for mechanical properties.

For the preparation of compounds Z1 and Z5, the guanidine G1 and G5, respectively, was liquefied beforehand at 60° C.

The results are shown in tables 1 and 2.

TABLE 1

| | | | | Viscosity | | | |
|---|---|---|---|---|---|---|---|
| Compo- | | | Concen- | [Pa · s] | | HBZ | |
| sition | Catalyst | Amount | tration¹ | fresh | stored² | fresh | stored² |
| Z1 | Guanidine | 1.69 g | 1.9 | 42.7 | 53.1 | 11′ | 9′ |
| | G1 | | | | | | |
| Z2 | Guanidine | 1.57 g | 1.9 | 41.7 | 54.8 | 22′ | 14′ |
| | G2 | | | | | | |
| Z3 | Guanidine | 1.66 g | 1.9 | 50.0 | 40.2 | 14′ | 21′ |
| | G3 | | | | | | |
| Z4 | Guanidine | 1.72 g | 1.9 | 30.2 | 27.5 | 24′ | 27′ |
| | G4 | | | | | | |
| Z5 | Guanidine | 1.67 g | 1.9 | 46.1 | 58.0 | 22′ | 33′ |
| | G5 | | | | | | |
| Z6 | Guanidine | 1.77 g | 1.9 | 32.2 | 30.4 | 23′ | 15′ |
| | G6 | | | | | | |
| Z7 | Guanidine | 1.84 g | 1.9 | 31.1 | 22.9 | 21′ | 21′ |
| | G7 | | | | | | |
| Z8 | DBU | 0.30 g | — | 26.3 | 31.0 | 29′ | 31′ |
| (Ref) | | | | | | | |
| Z9 | TMG | 0.22 g | 1.9 | 22.3 | 24.6 | 65′ | 75′ |
| (Ref) | | | | | | | |

¹mmol of guanidine groups per 100 g of polyether having silane groups.
²for 7 days at 70° C. in a closed container.

TABLE 2

| | Surface | Tensile | Elongation | Modulus of elasticity | |
|---|---|---|---|---|---|
| Composition | after 24 h | strength | at break | 0-5% | 0-50% |
| Z1 | dry | 0.75 MPa | 101% | 1.1 MPa | 0.8 MPa |
| Z2 | dry | 0.76 MPa | 101% | 1.1 MPa | 0.8 MPa |
| Z3 | dry | 0.78 MPa | 111% | 1.1 MPa | 0.8 MPa |
| Z4 | dry | 0.83 MPa | 123% | 1.1 MPa | 0.8 MPa |
| Z5 | dry | 0.89 MPa | 136% | 1.1 MPa | 0.8 MPa |
| Z6 | dry | 0.83 MPa | 126% | 1.2 MPa | 0.8 MPa |
| Z7 | dry | 0.78 MPa | 114% | 1.2 MPa | 0.8 MPa |
| Z8 (Ref) | greasy | 0.70 MPa | 95% | 1.0 MPa | 0.8 MPa |
| Z9 (Ref) | tacky | 0.62 MPa | 90% | 1.2 MPa | 0.8 MPa |

### Compositions Z10 to Z14:

In a planetary mixer, 36.2 g of polymer STP-2, 60.2 g of ground chalk (Omyacarb® 5 GU, from Omya), 1.2 g of thixotropic paste, the preparation of which is described hereinafter, 1.2 g of vinyltriethoxysilane, 1.2 g of 3-aminopropyltriethoxysilane and various catalysts in the amount specified according to table 3 were blended, and the mixture was tested as described for composition Z1 for viscosity, skin time (HBZ), surface characteristics and mechanical properties. The results are shown in tables 3 and 4.

The thixotropic paste was prepared by gently heating an initial charge of 300 g of diisodecyl phthalate (Palatinol® Z, from BASF) and 48 g of 4,4′-methylene diphenyl diisocyanate (Desmodur® 44 MC L, from Covestro) in a vacuum mixer and then slowly adding 27 g of n-butylamine dropwise while stirring vigorously.

The resultant paste was stirred for a further hour under reduced pressure while cooling.

TABLE 3

| | | | | Viscosity | | | |
|---|---|---|---|---|---|---|---|
| Compo- | | | Concen- | [Pa · s] | | HBZ | |
| sition | Catalyst | Amount | tration¹ | fresh | stored² | fresh | stored² |
| Z10 | Guanidine | 2.33 g | 2.6 | 174 | 145 | 3 h | 3 h |
| | G3 | | | | | 2′ | 32′ |
| Z11 | Guanidine | 2.41 g | 2.6 | 162 | 140 | 3 h | 2 h |
| | G4 | | | | | 19′ | 49′ |
| Z12 | Guanidine | 2.48 g | 2.6 | 167 | 143 | 1 h | 2 h |
| | G6 | | | | | 44′ | 11′ |
| Z13 | Guanidine | 2.58 g | 2.6 | 165 | 133 | 2 h | 2 h |
| | G7 | | | | | 30′ | 41′ |
| Z14 | DBU | 0.40 g | — | n.d. | n.d. | 1 h | n.d. |
| (Ref) | | | | | | 23′ | |

¹mmol of guanidine groups per 100 g of composition.
²for 7 days at 70° C. in a closed container.

TABLE 4

| | Surface | Tensile | Elongation | Modulus of elasticity | |
|---|---|---|---|---|---|
| Composition | after 24 h | strength | at break | 0-5% | 0-50% |
| Z10 | dry | 3.1 MPa | 145% | 4.0 MPa | 2.6 MPa |
| Z11 | dry | 2.8 MPa | 137% | 5.1 MPa | 2.8 MPa |
| Z12 | dry | 2.9 MPa | 142% | 4.7 MPa | 2.8 MPa |
| Z13 | dry | 2.7 MPa | 123% | 4.5 MPa | 2.7 MPa |
| Z14 (Ref) | greasy | 2.5 MPa | 155% | 4.0 MPa | 2.0 MPa |

1. A guanidine of the formula (I) 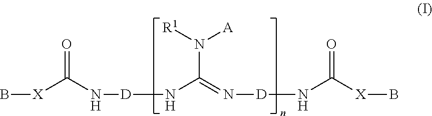
where
n is an integer from 1 to 20,
A is an alkyl, cycloalkyl or aralkyl radical which has 1 to 100 carbon atoms and optionally has one or more heteroatoms, or together with R¹ is an alkylene radical which has 2 to 6 carbon atoms and optionally contains ether oxygen or amine nitrogen and is optionally substituted,
R¹ is a hydrogen radical or is an alkyl radical which has 1 to 8 carbon atoms and optionally contains ether oxygen, or together with A is an alkylene radical which has 2 to 6 carbon atoms and optionally contains ether oxygen or amine nitrogen and is optionally substituted,
D is a divalent aliphatic or cycloaliphatic or arylaliphatic radical having 6 to carbon atoms,
B is a monovalent organic radical which has an average molecular weight in the range from 150 to 5,000 g/mol and is free of hydroxyl groups, primary and secondary amino groups and mercapto groups, and
X is O or S or NR² where R² is a hydrogen radical or is an alkyl radical which has 1 to 8 carbon atoms and optionally contains ether oxygen or tertiary amine nitrogen.

2. A guanidine as claimed in claim 1, wherein A is selected from the group consisting of methyl, ethyl, propyl, isopropyl, n-butyl, isobutyl, sec-butyl, tert-butyl, n-pentyl, isopentyl, n-hexyl, isohexyl, n-octyl, 2-ethylhexyl, n-decyl, lauryl, cocoalkyl, oleyl, cyclohexyl, benzyl, 2-methoxyethyl, 3-methoxypropyl, 3-(2-ethylhexyloxy)propyl, 3-(N,N-dimethylamino)propyl and ω-(alkyloxy)poly(oxyalkylene)alkyl having 1,2-oxyethylene and 1,2-oxypropylene units and a molecular weight in the range from 180 to 600 g/mol.

3. A guanidine as claimed in claim 1, wherein D is selected from the group consisting of 2-methyl-1,5-pentylene, 1,6-hexylene, 2,2(4),4-trimethyl-1,6-hexamethylene, (1,5,5-trimethylcyclohexan-1-yl)methane-1,3, 1,3-cyclohexylenebis(methylene), 1,4-cyclohexylenebis(methylene), 1,3-phenylenebis(methylene) and (methylenedicyclohexyl)-4,4′.

4. A guanidine as claimed in claim 1, wherein X is O and B is 2-(ω-(alkyloxy)poly(oxyethylene))ethyl,
2-(ω-(alkyloxy)poly(oxyethyleneoxypropylene))ethyl,
2-(ω-(alkyloxy)poly(oxyethyleneoxypropylene))propyl or
2-(ω-(alkyloxy)poly(oxypropylene))propyl, where alkyl is methyl, ethyl, butyl, 2-ethylhexyl, lauryl, cocoalkyl or oleyl and these radicals have a molecular weight in the range from 150 to 2,000 g/mol.

5. A guanidine as claimed in claim 1, wherein it has an average value of n in the range from 1.1 to 6.

6. A process for preparing a guanidine as claimed in any of claim 1 wherein a carbodiimide adduct of the formula (II) is reacted with at least one amine of the formula (III).

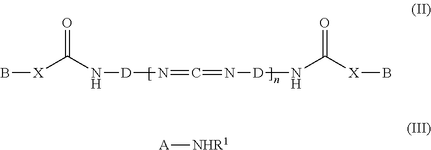

7. The process as claimed in claim 6, wherein
first at least one diisocyanate of the formula D-(NCO)₂ is converted in the presence of a carbodiimidization catalyst while heating and with release of CO₂,
then the resultant carbodiimide of the formula (IV) is reacted with at least one compound of the formula (V), 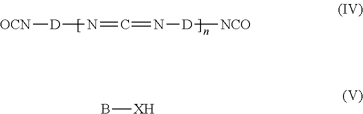
and finally the resultant carbodiimide adduct of the formula (II) is reacted with at least one amine of the formula (III).

8. A method comprising a step of catalyzing the crosslinking of a functional compound with the guanidine of claim 1.

9. The method as claimed in claim 8, wherein the functional compound is a silane, a polymer having silane groups, a polyisocyanate or a polyurethane polymer having isocyanate groups.

10. The method as claimed in claim 8, wherein the functional compound is a polyether having silane groups.

11. A curable composition comprising at least one guanidine as claimed in claim 1.

12. The curable composition as claimed in claim 11, wherein it contains silane groups and/or isocyanate groups.

13. The curable composition as claimed in claim 11, wherein it comprises at least one polymer having silane groups.

14. The curable composition as claimed in claim 11, wherein it is an adhesive or a sealant or a coating.

15. A cured composition obtained from the curable composition as claimed in claim 11.
